(19) United States
(12) Patent Application Publication
(10) Pub. No.: US 2021/0275827 A1
(43) Pub. Date: Sep. 9, 2021

(54) SYSTEMS AND METHODOLOGIES FOR TREATING OR PREVENTING PSYCHIATRIC DISORDERS, BRAIN TRAUMA, AND ADDICTION OR DEPENDENCE BY LIGHT THERAPY WITH MODULATED FREQUENCY

(71) Applicant: Reversal Solutions, Inc., Berkeley, CA (US)

(72) Inventors: Annelise E. Barron, Redwood City, CA (US); John A. Fortkort, Austin, TX (US); Peter A. Newsom, La Honda, CA (US); Jovanka Y. Hedeker, Berkeley, CA (US); Thomas A. Cocotis, Huntington Beach, CA (US); Robert R. Mullen, III, Woodland Hills, CA (US)

(21) Appl. No.: 17/195,068

(22) Filed: Mar. 8, 2021

Related U.S. Application Data
(60) Provisional application No. 62/986,677, filed on Mar. 7, 2020.

(51) Int. Cl. A61N 5/06

(52) U.S. Cl. CPC A61N 5/0618; A61N 2005/0642; A61N 2005/0626; A61N 2005/0659; A61N 2005/0663; A61N 2005/0652

(57) ABSTRACT

A method is provided for treating a subject for clinical depression or major depression, traumatic brain injury, opioid addiction, or other such physiological of psychological condition. The method includes diagnosing the subject as suffering from the condition; providing a light therapy unit comprising (a) a chassis, (b) a plurality of LEDs disposed on said chassis, and (c) a controller which controls the operation of said LEDs; and operating the plurality of LEDs at a plurality of distinct wavelengths of light such that the light emitted by the LEDs impinges on the subject, and such that the intensity of at least one of the plurality of distinct wavelengths of light is modulated at a frequency within the range of about 20 Hz to about 60 Hz.

### CROSS-REFERENCE TO RELATED APPLICATION

This application claims the benefit of priority from U.S. provisional application No. 62/986,677, filed Mar. 7, 2020, having the same inventorship, entitled “SYSTEMS AND METHODOLOGIES FOR TREATING OR PREVENTING PSYCHIATRIC DISORDERS, BRAIN TRAUMA, AND ADDICTION OR DEPENDENCE BY LIGHT THERAPY WITH MODULATED FREQUENCY”, and which is incorporated herein by reference in its entirety.

### FIELD OF THE DISCLOSURE

The present application relates generally to light therapy, and more specifically to methods for treating certain psychological or physiological conditions with a light therapy unit that operates at multiple wavelengths and utilizes selective frequency oscillation.

### BACKGROUND OF THE DISCLOSURE

Photobiomodulation therapy (PBMT) is a type of light therapy that utilizes non-ionizing electromagnetic energy to trigger photochemical changes in cellular structures that are receptive to photons. Various devices have been developed in the art to implement PBMT or processes related thereto. Examples of such devices are described, for example, in U.S. 2019/0246463A1 (Williams et al.)., U.S. US2019/0175936 (Gretz et al.), WO2019/053625 (Lim), U.S. U.S. 2014/0243933 (Ginggen), U.S. 2019/0142636 (Tedford et al.), U.S. Pat. No. 7,354,432 (Eells et al.), U.S. 2008/0091249 (Wang), U.S. Pat. No. 10,391,330 (Bourke et al.) and U.S. 2016/0129278 (Mayer).

Although the effects of PBMT are not fully understood, the underlying physiological processes at play during PBMT have been the subject of considerable research. Mitochondria are central to these processes. These intracellular organelles generate adenosine triphosphate (ATP), which is the main source energy for cellular activity and metabolism.

Mitochondria absorb visible red and near infrared light (NIR) energy at the cellular level, and utilize the absorbed radiation to produce cellular energy in the form of ATP. A mitochondrial enzyme (cytochrome oxidase c) is central to this process. This enzyme is a chromophore, and accepts photonic energy of specific wavelengths when functioning below par.

The process utilized by mitochondria to generate ATP also creates reactive oxygen species (ROS). These species promote gene transcription, cellular repair and healing. This process is also believed to release nitric oxide back into the body. Nitric oxide helps cells to communicate with each other, and also improves blood circulation and dilates blood vessels.

### BRIEF DESCRIPTION OF THE DRAWINGS

FIGS. 1-2 are illustrations of a light therapy system which may be utilized to implement some of the methodologies disclosed herein.

FIG. 3 depicts the base portion of the device of the device of FIGS. 1-2.

FIG. 4 depicts a remote control that may be utilized to control the device of FIGS. 1-2.

### SUMMARY OF THE DISCLOSURE

In one aspect, a method is provided for treating a subject for depression. The method comprises (a) diagnosing the subject as suffering from clinical depression or major depression; (b) providing a light therapy unit comprising (i) a chassis, (ii) a plurality of LEDs disposed on said chassis, and (iii) a controller which controls the operation of said LEDs; and (c) operating the plurality of LEDs at a plurality of distinct wavelengths of light such that the light emitted by the LEDs impinges on the subject, and such that the intensity of at least one of the plurality of distinct wavelengths of light is modulated at a frequency within the range of about 20 Hz to about 60 Hz.

In another aspect, a method is provided for treating a subject suffering from a traumatic brain injury. The method comprises (a) diagnosing the subject as suffering from a traumatic brain injury; (b) providing a light therapy unit comprising (i) a chassis, (ii) a plurality of LEDs disposed on said chassis, and (iii) a controller which controls the operation of said LEDs; and (c) operating the plurality of LEDs at a plurality of distinct wavelengths of light such that the light emitted by the LEDs impinges on the subject, and such that the intensity of at least one of the plurality of distinct wavelengths of light is modulated at a frequency within the range of about 20 Hz to about 60 Hz.

In a further aspect, a method is provided for treating a subject suffering from an opioid addiction. The method comprises (a) diagnosing the subject as suffering from an opioid addiction; (b) providing a light therapy unit comprising (i) a chassis, (ii) a plurality of LEDs disposed on said chassis, and (iii) a controller which controls the operation of said LEDs; and (c) operating the plurality of LEDs at a plurality of distinct wavelengths of light such that the light emitted by the LEDs impinges on the subject, and such that the intensity of at least one of the plurality of distinct wavelengths of light is modulated at a frequency within the range of about 20 Hz to about 60 Hz.

In still another aspect, a method is provided for treating a subject suffering from ADHD. The method comprises (a) diagnosing the subject as suffering from ADHD; (b) providing a light therapy unit comprising (i) a chassis, (ii) a plurality of LEDs disposed on said chassis, and (iii) a controller which controls the operation of said LEDs; and (c) operating the plurality of LEDs at a plurality of distinct wavelengths of light such that the light emitted by the LEDs impinges on the subject, and such that the intensity of at least one of the plurality of distinct wavelengths of light is modulated at a frequency within the range of about 20 Hz to about 60 Hz.

In yet another aspect, a method is provided for treating a subject suffering from PTSD. The method comprises (a) diagnosing the subject as suffering from PTSD; (b) providing a light therapy unit comprising (i) a chassis, (ii) a plurality of LEDs disposed on said chassis, and (iii) a controller which controls the operation of said LEDs; and (c) operating the plurality of LEDs at a plurality of distinct wavelengths of light such that the light emitted by the LEDs impinges on the subject, and such that the intensity of at least one of the plurality of distinct wavelengths of light is modulated at a frequency within the range of about 20 Hz to about 60 Hz.

In still other embodiments, methods are provided for preventing the development of conditions such as depression, addiction, ADHD, PTSD, or condition resulting from traumatic brain injury. These methods include providing a light therapy unit comprising (i) a chassis, (ii) a plurality of LEDs disposed on said chassis, and (iii) a controller which controls the operation of said LEDs; and operating the plurality of LEDs at a plurality of distinct wavelengths of light such that the light emitted by the LEDs impinges on the subject, and such that the intensity of at least one of the plurality of distinct wavelengths of light is modulated at a frequency within the range of about 20 Hz to about 60 Hz.

In any of the foregoing embodiments, one or more audio tracks or audio files may be provided that are coordinated and/or synchronized with the plurality of LEDs or the light emitted therefrom. Preferably, the audio tracks or audio files include sound that is modulated, coordinated and/or synchronized with the LEDs or the light emitted therefrom at a frequency within the range of about 20 Hz to about 60 Hz.

### DETAILED DESCRIPTION

### A. Overview

Some benefits of photobiomodulation therapy (PBMT) have been recognized in the art. For example, a recent study by Iaccarino et al. (see Iaccarino, H. F., Singer, A. C., Martorell, A. J., Rudenko, A., Gao, F., Gillingham, T. Z., . . . Tsai, L. H. (2016), “Gamma Frequency Entrainment Attenuates Amyloid Load and Modifies Microglia”, Nature, 540(7632), 230-235) indicated that the non-invasive method of flickering light which is disclosed therein may induce gamma waves in the brain, and may reduce pathological symptoms of Alzheimer's disease (AD). In this study, mice that were genetically engineered to develop AD were exposed to an LED light source that flickered at 40 Hertz (Hz). After treatment with the oscillating light source for one hour, the mice showed reduction of amyloid beta (Aβ) plaques levels in the visual cortex by half (Aβ plaques are known to be associated with AD). In another study (see Köster, M.et al. (2019), “Memory Entrainment by Visually Evoked Theta—Gamma Coupling”, Neuroimage188, 181-18785), visual theta stimulation was found to lead to enhanced memory performance. Despite these studies, however, many of the possible effects of PBMT remain unknown.

It has now been found that light therapy in general, and PBMT in particular, may be utilized as an effective tool in treating a subject for certain psychological or physiological conditions, in lessening the severity or effects of these conditions, and/or in preventing the occurrence of these conditions. These conditions include, but are not limited to, traumatic brain injury, addiction or dependence (including, for example, addiction to, or dependence on, opioids, amphetamines, stimulants, alcohol or cannabis), depression (and more specifically, clinical depression or major depression), PTSD, ADHD, and developmental trauma disorder, traumatic brain injury and its sequelae.

In a preferred embodiment of the methodologies disclosed herein, a subject is diagnosed as suffering from at least one of the foregoing conditions. A light therapy unit is then provided which preferably comprises (a) a chassis, (b) a plurality of LEDs disposed on the chassis, and (c) a controller which controls the operation of said LEDs. The light therapy unit is then positioned in a therapeutically effective orientation with respect to the subject (or alternatively, the subject is positioned in a therapeutically effective orientation with respect to the light therapy unit). The plurality of LEDs is then operated at one or more of first, second and third distinct wavelengths of light (and preferably at red, near-infrared and blue-turquoise wavelengths) such that the light emitted by the LEDs impinges on the subject. In some embodiments, one or more of the wavelengths of light may be modulated in their intensity at a frequency within the range of about 20 Hz to about 60 Hz, and more preferably are modulated in their intensity at about 40 Hz. In some embodiments, the light therapy may be accompanied by one or more music or audio files, which may include a track or portion thereof which is modulated at a frequency within the range of about 20 Hz to about 60 Hz, and more preferably is modulated at about 40 Hz.

### B. Exemplary Embodiment

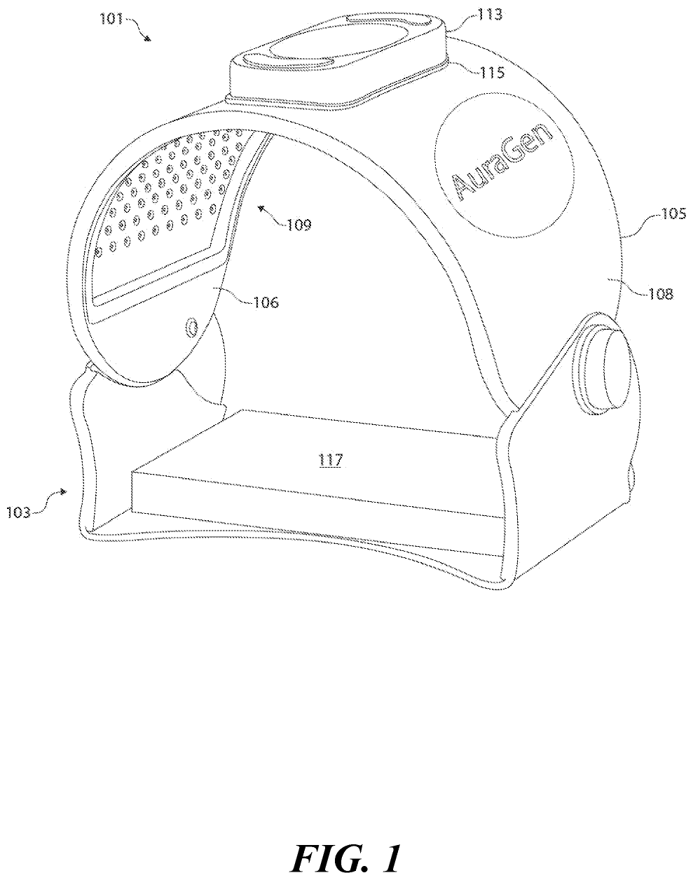

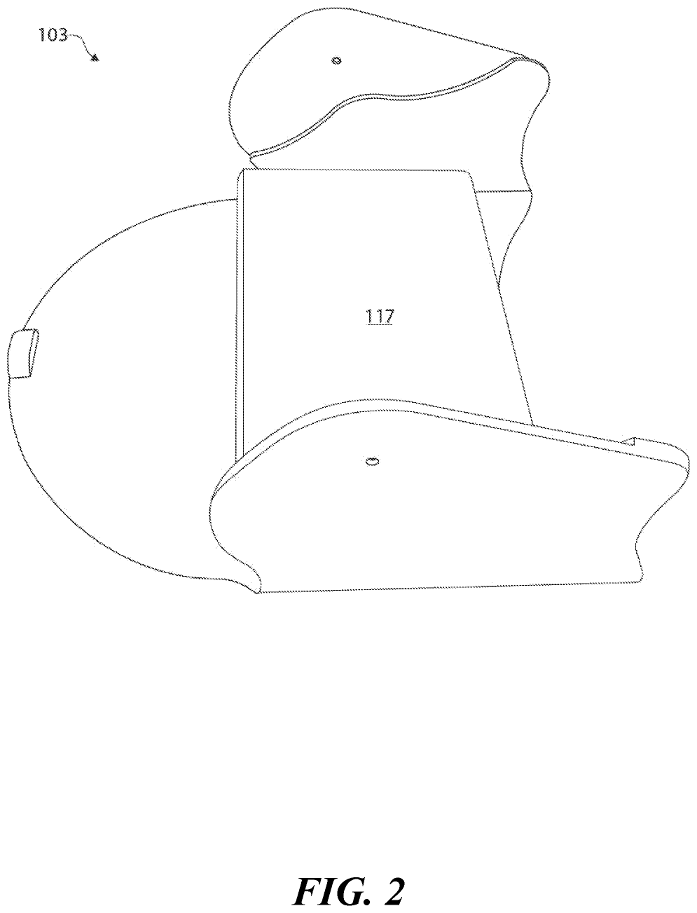

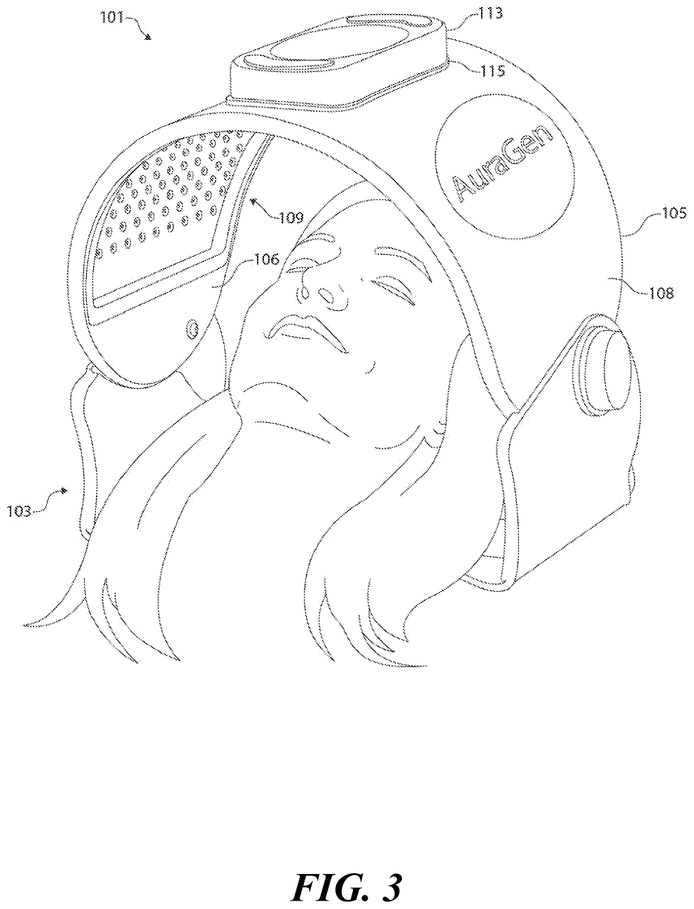

FIG. 1-3 depict a first particular, non-limiting embodiment of a device in accordance with the teachings herein. With reference thereto, a light therapy device 101 is provided which comprises a base 103 (shown in isolation in FIG. 2) having a peripheral element 105 attached thereto and, optionally, an audio headset (not shown; the need for a headset may be determined, for example, by whether the entrainment methodology uses traveling waves originating from the same source, or standing waves generated by two distinct sources). The base 103 and peripheral element 105 define an opening 107 in which a user's head is placed (see FIG. 3). The base 103 and/or peripheral element 105 may be equipped with an audio jack, a Bluetooth transmitter, or other suitable provisions as necessary or desirable to support the use of an audio headset by the user. The base 103 is also equipped with a pillow 117 to support the head of the user.

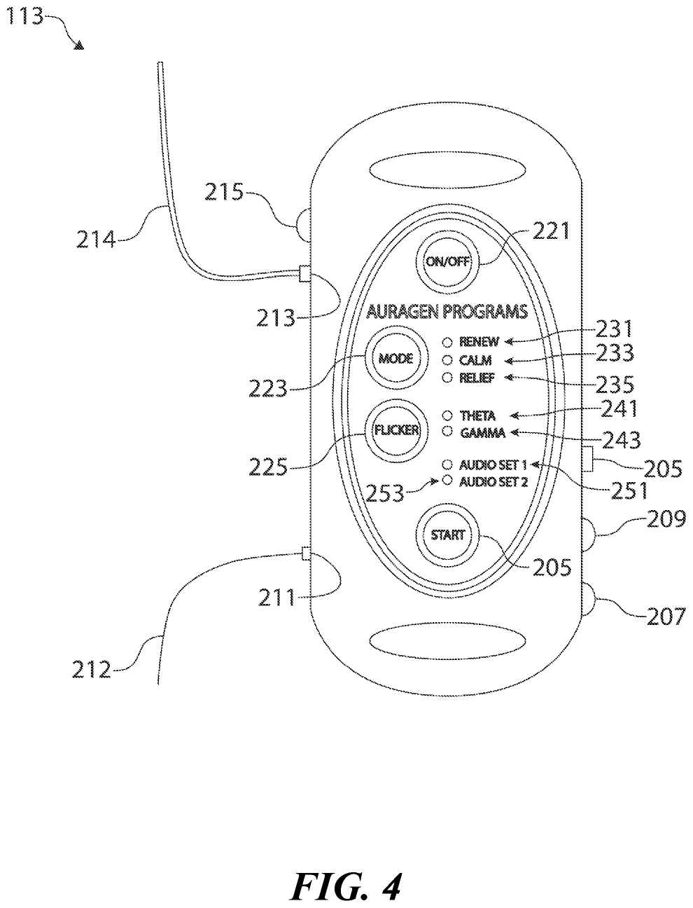

The base 103 in this particular embodiment is equipped with a pillow 111 for user comfort, and to provide the user with the ability to lie down or sleep during a brainwave entrainment session. The peripheral element 105 has a first major inward-facing surface 106 and a second major outward-facing surface 108. The first major surface 106 is equipped with an LED array 109 which can be activated with a remote control 113 to illuminate the user's head at one or more wavelengths. The second major surface 108 is equipped with a holder 115 for the remote control 113. The remote control 113, which is shown in greater detail in FIG. 4, may also be utilized to modulate the light emitted by the LED array 109, to select one or more wavelengths of light emitted by the LED array 109, and to control the playback of one or more audio files or tracks.

In normal use, a user's head is placed in the opening 107 such that the back of the user's head is on the pillow 111 and such that the user is facing the first major surface 106 of the peripheral portion 105 as shown in FIG. 21. The user (or possibly a clinician or other assistant) then uses the remote control 113 to activate the light therapy device 101 and to cause it to function in one or more selected modes. Regarding the latter, it is to be noted that the light therapy device 101 may be programmed with various algorithms which cause it to function in particular ways, some of which are described in greater detail below. The light therapy device 101 may also be programmed to play music or soundtracks, which may be advantageously matched to the particular algorithm being implemented by the light therapy device 101.

In some embodiments, the entrainment device may include a port to allow plugin of additional LED portable devices that operate in concert with the light therapy device 101 to provide light therapy to specific parts of the body. For example, such a portable LED device may be adapted to be positioned in the mouth of the user (via, for example, a mouth guard). In other embodiments, the entrainment device may include a small pad that may be wrapped or directly applied to a specific body part of the user. In still other embodiments, the entrainment device may include a set of googles or glasses that are placed over the eyes of the user to provide focused treatment to those areas, or to prevent treatment of those areas. Of course, it will be appreciated that any of the foregoing accessories may be utilized in combination in various embodiments of the systems and methodologies disclosed herein.

Various LEDs 109 or other light sources which emit at various wavelengths may be utilized in the devices and methodologies disclosed herein. However, the use of light sources which emit at wavelengths in the red, infra-red and blue-turquoise regions of the spectrum are preferred, and the use of light sources which emit at about 470 nm, 670 nm and 870 nm are especially preferred. In a preferred mode of operation, these light sources are made to oscillate or flicker in the theta or gamma band.

It will be appreciated that light may be emitted at the foregoing wavelengths in various manners, including sequentially or simultaneously. For example, the LED array 109 may be operated to emit electromagnetic radiation at a single wavelength (i.e., monochromatically) or at multiple wavelengths. In some cases, the LED array 109 may include a first set of LEDs that are operated to emit light at a first wavelength, a second set of LEDs that are operated to emit light at a second wavelength, and (optionally) a third set of LEDs that are operated to emit light at a third wavelength. In other cases, the LED array 109 may be operated such that all of the LEDs in the array emit light at a first wavelength for a first period of time, all of the LEDs in the array emit light at a second wavelength for a second period of time, and (optionally) all of the LEDs in the array emit light at a third wavelength for a third period of time.

The particular wavelength(s) of emission of the LED array 109, the duration of those emissions, the frequency of oscillation (if any), the intensity of the emitted light, the selection of accompanying audio tracks or files (if any), and/or the oscillation of any accompanying audio tracks, files or component(s) thereof, may be selected to achieve a desired physiological or psychological effect. It will be appreciated that, in some embodiments, the duration of emission for any particular wavelength of light may remain constant or may vary during the course of a therapy session. It will further be appreciated that, in some embodiments, any of the LEDs in the LED array 109 may be operated to emit two or more wavelengths of light, including broadband radiation or white light.

FIG. 4 depicts a particular, non-limiting embodiment of a remote control 113 that may be utilized with the light therapy device 101 of FIGS. 1-2. The remote control 113 comprises a body 201 which houses the electronics of the remote control 113, which will typically include an appropriate chipset and other suitable control circuitry. The remote control 113 is equipped with a central keypad 203 and peripheral controls, the latter of which include a track selection 205 for selecting one of a plurality of prerecorded audio tracks, a first volume control 207 for adjusting the audio volume of the selected audio track, and a second volume control 209 for controlling the volume of a second soundtrack featuring a sound at a specific frequency (for example, a gamma or beta frequency), which may be a diurnal beat. The two soundtracks may be played together or independently of each other.

The remote control 113 is further equipped with a headset audio plug-in port 211 for connecting a wired headset 212 to the remote control 113, and a power plug-in port 213 for connecting a power cord 214 to the remote control 113. The power cord 214 may be utilized to power the remote control 113 or to recharge one or more internal batteries contained within the device. The remote control 113 is also equipped with an LED indicator 215 to indicate when it is in a powered-on state.

The central keypad 203 includes an on/off button 221 which turns the remote control 113 on and off. A mode button 223 allows the user to toggle among mode selections (here, “Renew” 331, “Calm” 333 and “Relief” 335 mode selections), wherein each mode operates the light therapy device 101 in accordance with a particular program. A flicker button 225 allows the user to toggle among flicker settings. In the particular embodiment depicted, the flicker button 225 allows the user to select flickering at theta 241 or gamma 243 frequencies, or to deactivate flickering altogether. In the particular embodiment depicted, the central keypad 203 also includes audio set indicators which track which of a plurality of audio sets (here, audio set 1 251 and audio set 2 253) the track selection button 205 is sampling audio tracks from.

C. Conditions Treatable with Light Therapy

As previously noted, it has been found that PBMT may be utilized as an effective tool in treating a subject for certain psychological or physiological conditions, or for prevention of these conditions. These conditions include, but are not limited to, traumatic brain injury, addiction or dependence (including, for example, addiction to, or dependence on, opioids, amphetamines, stimulants, alcohol or cannabis), depression (and more specifically, clinical depression or major depression), PTSD, developmental trauma disorder, and traumatic brain injury and its sequelae. In a preferred embodiment of the methodology disclosed herein, a subject is first diagnosed as suffering from one of the foregoing conditions, and then PBMT is utilized to treat the subject.

### C1. Depression

As previously noted, the devices and methodologies disclosed herein may be utilized to treat depression, and especially clinical depression or major depression. Depression is a common disorder with a lifetime incidence on the order of 10 to 20%. Depression affects about 8% of women and 4% of men. An additional, about equal number of the population is at risk for less severe forms of depression. Depression affects all social classes and all ethnic groups, and is worldwide in distribution.

Over the course of the past century, depression appears to be occurring earlier in lifespans on average (this has been labeled the cohort effect). Depression also appears to affect a higher percentage of each subsequent generation (this has been called the period effect). The most severe forms of depression (such as, for example, melancholia) are less common, and affect about 1-2% of the general population.

Many instances of depression are unipolar, meaning these people either live in a normal state or they are significantly depressed and can be said to have depression. A smaller percentage of depressed people have bipolar disorder. People with bipolar disorder may be in one of three different states: a normal state (called euthymic), a depressed state (called depression), or an elevated state (known as mania). Many patients with bipolar depression are thought to have ordinary unipolar depression until the first episode of mania occurs; after this, the main diagnosis changes to bipolar, and their subsequent depressions are called bipolar depression.

In dealing with a patient who appears to be sad and pessimistic, a psychiatrist must typically determine whether the patient is suffering from depression or demoralization. The latter designation means the patient is demoralized or disappointed, but the condition may be resolved within minutes of, for example, receiving some good news. Only actual depression responds to antidepressant therapy, and depression is a much different process from demoralization. True depression absolutely cannot change within a matter of minutes, and on the contrary, typically takes weeks or months to fully resolve, even with treatment. Demoralization may certainly be a condition which may be precipitated by stressful life events, and a suite of these life events, whether traumatic or not, may increase the odds that a person will fall into a real depression. However, what really differentiates depression from demoralization is that, whereas patients suffering from both conditions feel their mood to be low or sad, the person with major depression has additional difficulties. These may include problems with sleep, motivation, concentration, sexual interest or desire, exhaustion, and declining function in general. These problems may be exhibited in work or in social situations.

According to the Diagnostic and Statistical Manual of Mental Disorders (DSM-5), classic depression is termed major depressive disorder and it has characteristics such as being depressed most of the day nearly every day, decreased motivation or interest or pleasure in all or almost all activities, significant weight loss when not dieting or weight gain, insomnia or hypersomnia, psychomotor agitation or retardation, fatigue nearly every day, feelings of worthlessness or excessive guilt, difficulty thinking, and recurrent thoughts of death. Associated features may include depressive pseudo-dementia, which is an extremely important diagnosis in the elderly. In particular, many elderly patients are misdiagnosed as having dementia or Alzheimer's disease, when they are actually having the cognitive effects of depression (and therefore, these cognitive effects are very definitely treatable).

Depression is also common in certain specific life situations. For example, depression may follow a period of prolonged grief or may occur during a postpartum period, when it is termed postpartum depression. Other forms of depression include atypical depression, premenstrual dysphoria, seasonal affective disorder, and less severe depressive conditions including, for example, adjustment disorders. Depressions of all types appear to result from multiple causes. Some of these causes may be unknown, and some appear to be due to well-known factors (such as, for example, certain medications including antihypertensives, antiarrhythmics, anticonvulsants, antibiotics, and hormonal agents). Hormonal causes may include thyroid diseases, hyperparathyroidism, Cushing's disease, and subclinical thyroid disorders. The latter includes situations wherein thyroid hormones are officially within “normal” limits, but are not at optimal levels (i.e., not in the higher quartile of the normal bell curve). Many other medical conditions may cause depression, including cerebrovascular disease, stroke, cancer, fibromyalgia, and Parkinson's disorder.

Treatment of depression typically includes psychotherapy, which may involve individual or family therapy, medication management, and more severe measures such as, for example, transcranial magnetic stimulation and electroconvulsive therapy (ECT). In cases of mild depression, talk therapy may be all that is necessary, providing a chance for the patient to speak to an encouraging and sympathetic (and hopefully knowledgeable) therapist.

There is a general consensus that, if the depression is more severe or is not responding to talk therapy alone, the next goal of treatment advised would be pharmacotherapy. This treatment utilizes antidepressants and, in some cases, auxiliary medications which can augment the effect of the antidepressant (these may include, for example, lithium, thyroid, or other psychopharmacological agents). Selective Serotonin Reuptake Inhibitors (SSRIs) are now one of the most common forms of pharmacotherapy. SSRIs include, without limitation, fluoxetine and paroxetine. Other agents currently in use include venlafaxine and Cymbalta, and older agents including tricyclic antidepressants.

Family therapy may also be extremely useful in treating depression. It is especially important to encourage the family to understand that the patient is not malingering (i.e., is not lazy), but rather has a significant medical illness of the brain. It is also important for the family to appreciate that the manner in which the illness is regarded by family members plays a significant role in helping the patient to recover. It is essential that family members understand that the patient is already doing all they can, and is not simply laying down on the job.

More severe forms of depression, such as melancholia or melancholic depression, often need more severe measures. These may include transcranial magnetic stimulation and electroconvulsive therapy (ECT).

### C2. Posttraumatic Stress Disorder

As previously noted, the devices and methodologies disclosed herein may be utilized to treat posttraumatic stress disorder (PTSD). PTSD is a stress disorder caused by experiencing or witnessing a traumatizing event of sufficient severity (such as, for example, sexual violence, or a significant bodily injury to oneself or to another). The patient then appears to suffer a very real and prolonged reaction to this stressful event, which is described in DSM-5 as follows.

First of all, there must first be exposure to an unequivocally traumatizing event, such as death, serious injury or sexual violence. This event must be directly experienced (that is, witnessed in person as the event occurs to oneself or others). The event then may be more traumatic if it is learned that it has occurred to a close family member or close friend and, in the case of actual or threatened death to family member or friend, the event must have been violent or accidental. Experiencing repeated and extreme forms of exposure to aversive details also puts people more at risk (such as seen in first responders).

Secondly, the event must be re-experienced in a repetitive and persistent fashion, either in one's thoughts, or in images or dreams. These events are described as nightmares, recurrent discussing dreams, distressing memories, flashbacks, or prolonged distress at exposure to internal or external cues. In the case of children, repetitive play may occur along traumatic themes, and the patient may also have frightening dreams without recognizable content.

Thirdly, the victim must make an effort to avoid any stimuli which are associated with the trauma. This may include avoiding thoughts or feelings associated with the events, or avoiding people or conversations associated with the event.

Fourthly, the victim must demonstrate different forms of increased arousal which were not previously present. These may include, for example, insomnia, irritable or angry outbursts, hypervigilance, reckless or self-destructive behavior, poor concentration, or exaggerated startle response.

Fifthly, the victim must have an inability to recall important parts of the trauma. The victim must also have persistent and exaggerated negative beliefs or expectations about self, others or the world. The victim must further have persistent distorted cognitions about the cause or consequences of the event. The victim must also have persistent negative emotional states. The victim must also have a sense of a foreshortened future. Finally, the victim must have feelings of detachment or estrangement from others.

Sixthly, the effects of this disorder must be persistent, and must affect the victim seriously enough that it significantly and negatively affects the social life, job performance, or interpersonal relations of the victim.

The treatment of PTSD typically includes psychopharmacological medication and various forms of psychotherapy, including individual and group forms of therapy. Psychopharmacological medications as currently employed are not specific to PTSD per se. Rather, medication is used on an ad hoc basis as symptoms arise in the course of treatment. For instance, one very frequent long-term symptom of PTSD is insomnia, which may be chronic and severe. Medications such as Inderal or prazosin, which may also be augmented with further hypnotic agents such as Ambien or benzodiazepines, can be used as the need arises so that the patient is at least receiving eight hours of sleep. Another frequent symptom of PTSD is depression, which at times may be severe and may even lead to suicidal thoughts or behaviors. In the event of significant depression complicating the PTSD, psychopharmacological medication for depression (such as, for example, antidepressants, mood stabilizers, lithium, and other medications) may be used in order to help the patient achieve remission from depression. This may have very significant positive effects such as giving the patient the mental energy needed to undergo the (at times stressful) forms of therapy specific to PTSD, the most common of which is Eye Movement Desensitization and Re-programming (EMDR).

The general efficiency of treatment for PTSD is dependent upon a number of factors. These may include, for example: (1) any complicating psychological factors such as depression, anxiety, panic, insomnia; (2) the chronicity of the PTSD; (3) other factors that are frequently associated with the any coexisting associated physical injuries (i.e., any symptoms caused by injuries which actually occurred at the time of the initial event) such as chronic pain or debilitating behavioral, psychological and/or cognitive affects as frequently seen in TBI or traumatic brain injury; (4) the appropriateness and availability of those treatments specifically designed for PTSD and found to be effective in treating it such as, for example, eye movement desensitization and reprocessing therapy (EMDR) and affective forms of group therapy which are designed with PTSD patients in mind; and (5) neuro feedback, which has been found to be quite affective based on the work of Sebern Fisher and others.

Drawbacks of current treatment include expense. In particular, EMDR tends to be performed by personnel (such as EMDR therapists or psychiatrists) who are specifically trained in this art, and this modality involves one-on-one sessions with such personnel which may go on for months or even years. Medication management is also a problem in that there are no medications specifically found to treat the overall condition of PTSD. Rather, medications are typically used on an ad hoc basis for whatever symptoms are significant enough to require treatment in order to prevent further deterioration.

### C3. Traumatic Brain Injury (TBI)

As previously noted, the devices and methodologies disclosed herein may be utilized to treat traumatic brain injuries.

Traumatic brain injury syndrome is a diagnostic term applied to an extremely varied group of chronic brain disorders having in common simply the etiology of being caused by a traumatic injury to the brain. These disorders are utterly protean in manifestation, however, at least in terms of their symptomatology. TBI syndrome often includes some common initial symptoms such as photophobia, confusion, difficulty concentrating, headaches, and an impression of not firing on all cylinders. Patients suffering from TBI often have difficulty comprehending or describing what is wrong or articulating the symptoms they are having, other than to say that they cannot function.

There is no DSM definition of TBI, and it is not listed in the DSM V per se. However, in 2009, the Department of Veterans Affairs and the Department of Defense proposed that TBI be defined as “a true medically induced structural injury and/or physiological description of brain function as a result of an external force that is indicated by at least one of the following clinical signs, immediately following the event:

(a) any period of loss or a decreased level of consciousness;

(b) any loss of memory for events immediately before or after the injury;

(c) any alteration in mental state at the time of the injury (confusion, disorientation, slowed thinking etc., also known as alteration of consciousness);

(d) neurological deficits (weakness, loss of balance, change in vision, praxis, paresis/paraplegia, sensory loss, aphasia, etc.) that may or may not be transient; or

(e) intracranial lesion.”

Traumatic brain injuries may be rated as mild, moderate or severe, depending on the results of initial testing to determine the functioning of a patient in the early hours after the occurrence. Functioning is rated by checking for some of the most common results of significant head injuries, such as loss of consciousness, confusion, feeling dazed, disoriented or confused, post traumatic amnesia, and other manifestations. These manifestations may be determined by physical exam or by interviewing the patient. Patient interviews may utilize lists of questions which have been tabulated in various standard TBI questionnaires. The Glasgow coma scale (GCS), a well-known and accepted modality for measuring level of consciousness, has been used as a way of rating the initial and subsequent severity of any head injury. However, the usefulness of the GCS in predicting the severity of the initial injury is felt to be greater for moderate and severe TBI, since the vast majority of TBI patients will have normal (or near normal) scores within hours after the injury. TBI is not excluded by a loss of consciousness (LOC) at time of impact, and mild TBI can occur without any LOC or PTA. Furthermore, the term “mild TBI” is used somewhat synonymously with the term concussion, meaning either a traumatic loss of consciousness, or a deficit in functioning (such as amnesia or any focal neurological deficit) in the aftermath of a blow to the head.

Common symptoms in the initial post injury period include photophobia, confusion and decreased concentration. Often the patient has only a very dim idea of why they are no longer the successful, capable or socially adept person they once were. This may lead to the loss of a patient's peer group, social group or friends, which may result in chronic despair or substance abuse. Over time, much more complex symptoms may also become more apparent. The patient's ability or inability to understand these sorts of deficits (such as cognitive deficits or loss of emotional regulation, for example) and to maintain a positive attitude about the necessary treatment may cause still other longer term deficits to occur. These other long term deficits may include the development of psychiatric conditions such as depression (if despair over the situation dominates the course), or intermittent explosive disorder (when the new emotional lability can start to appear almost like a new personality). This may become an iterative process as the patient becomes increasingly bitter and unwilling to do the work required, thus making the symptoms much more difficult to alleviate. Indeed, in the aftermath of the often slow discovery of just how deep and severe the deficits are, as well as the gradual discovery of the myriad difficulties that TBI has been causing the patient, TBI can lead to the new occurrence of virtually any axis one psychiatric diagnosis in the DSM.

Compounding the problems these patients already face, the diagnosis of TBI is frequently missed entirely or subsequently, especially if there was no initial and discernible loss of consciousness or obvious change in function. The availability of advanced diagnosis and treatment is also quite variable. Thankfully, this is now being addressed (at least in some large metropolitan areas) by the development of dedicated TBI specialty clinics. These facilities attempt to include all of the specialties needed for TBI care, including psychiatry, psychotherapy, cognitive therapy, and group therapy. These facilities also attempt to include very advanced radiographic imaging and other diagnostic technologies, including CT, MRI, Diffuser Tensor Imaging, ultrasound, quantitative EEG, and other modalities. Obviously, such complex and expensive technologies and such well manned and comprehensive treatment teams are simply not available in many small local hospitals and clinics, and the availability of clinicians skilled in the detection of the more subtle presentations remains spotty at best, even in many large metropolitan areas.

It is also important to understand the co-occurrence of PTSD with TBI. Such co-occurrence has been seen increasingly in the past two decades at the VA (especially in veterans returning with blast injuries from LEDs), although it is also a major part of civilian TBI morbidity. This co-occurrence leads to a confluence of being unable to function in life, being unable to understand why, and being unable to get good advice and enough compassionate understanding of the severity of the illness or enough peer support to help the TBI patient to stay on the long road to recovery. This may lead to a new form of severe and repetitive trauma, a sort of limbo existence which may become a living hell due to the absence of realistic hope in the patient's recovery process.

Fortunately, there are new and promising developments on the horizon. These include new medication practices featuring amantadine and prazosin, new group treatments for TBI and for the combination of TBI and PTSD, and the development of computer aided programs. Examples of the latter include the Cognitive Symptom Management and Rehabilitation Therapy (Cogsmart) program developed by a consortium of VA TBI treatment centers. This program is a form of cognitive training to help people improve their skills in prospective memory (remembering to do things), attention, learning/memory, and executive functioning (problem-solving, planning, organization, and cognitive flexibility).

There has been significant success in training TBI sufferers to function more optimally through the use of a number of new technologies, including quantitative EEG guided neurofeedback featuring slow or “infra” wave formats. These modalities have sometimes led to good results, but they have the drawback (in some cases) of requiring the very sorts of prolonged concentration and mental effort that TBI sufferers are frequently incapable of, at least during early stages of recovery. Therefore, it is quite promising that various, newly developed forms of light therapy have been found to offer either a replacement for Neurofeedback, or at least a bridge therapy. Light therapy has the distinct advantage that it is a passive therapy (that is, it demands little or no mental effort) and therefore can be used in situations common to TBI where mental effort is still so exhausting that such a therapy can be unsuccessful or can even be counterproductive (that is, it may do more harm than good). Light therapy also has the advantage of having no side effects when used correctly. It may be employed to treat TBI to great effect, including helping such diverse symptoms as poor memory and attention, insomnia, fatigue, mental fog, emotional dysregulation, irritability, inhibition, depression, anxiety, balance problems, hyperacusis, and severe PTSD symptoms. Importantly, it has also been shown to increase mental energy and focus (which could allow integration with active therapies such as neurofeedback) once the patient has healed enough to have the mental energy necessary to make these further treatment advances which neurofeedback can lead to.

The annual toll of significant head injuries in the U.S. by multiple estimates is between one and three million new cases a year. It is critical that these cases be identified as early as possible, that treatment start as early as possible, and that the patient have the needed support in order needed to accept the functional losses that have occurred. It is also important for the patient to be given a reasonable and accurately comprehensive diagnosis and prognoses, and to have an understanding of the tremendous amount of work that recovery may entail, as well as all the forms of peer support which have proved so necessary for sticking with the protocol long enough to get better. Far too often, these cases are neglected at the time of initial evaluation, which frequently results in the patient receiving vague diagnoses and even vaguer suggestions as to how long it will take to get function back to anywhere near normal. This frequently causes the patient to fall off the radar due to discouragement and resignation, thus resulting in further deterioration of the patient's condition.

It will be appreciated from the foregoing that all modalities of therapy, especially those without side effects such as light therapy, need to be made available to as many TBI sufferers as possible. Such an approach is required to turn the silent epidemic of TBI in the direction of effective therapy, and to prevent further morbidity as is seen so often today.

### C4. Attention Deficit Hyperactivity Disorder (ADHD)

As previously noted, the devices and methodologies disclosed herein may be utilized to treat ADHD.

ADHD is a DSM recognized diagnosis for a neurobiological disorder known to affect early development and school performance, as well as later employment capacities, family function, and possibly other widespread aspects of behavior. It is conservatively thought to have a prevalence of about 3-5% of school age children, with boys more affected than girls. ADHD is (to put it mildly) a problematic diagnosis. Originally estimated to affect approximately 3 to 9% of school-age children, the incidence of this comparatively recent diagnostic entity has mushroomed over the past 20 years to the point where many school districts are now reporting it in 30 to 35% of grammar school age boys. Keith Conner, who originally created the classic diagnostic symptom list, was so troubled at the overuse and overdiagnosis of this condition in the latter part of his career that he engaged in a campaign to alert the therapeutic and psychiatric community to the alarming extent to which he felt the diagnosis had become a victim of chronic overdiagnosis. This appears to be very much the case, and seems to have occurred early on in no small part due to advertising by the pharmaceutical industry. Later overusage of this diagnosis appears to have been caused by school districts eager for funding, and also by increasing numbers of students abusing the stimulant medication for goals such as grades, weight loss, or selling the drug illegally to other abusers.

ADHD, a diagnosis which was originally was thought to almost entirely concern school age children and adolescents, has since been found to persist into adulthood in at least 40 or 50% of affected individuals. Tragically, although the phenotypic presentation in preschoolers has not even been established (as Connor has described), the “diagnosis” of ADHD has now even been extended to preschoolers, and there are now reports of children as young as two being placed on stimulants with little or no research into what later effects this could lead to in the child.

There is also a very strong cross-diagnosis between ADHD and bipolar disorder, with individuals originally diagnosed has ADHD eventually also acquiring the diagnosis of bipolar disorder approximately 30 to 40% of the time. Similarly, those individuals originally diagnosed as bipolar also eventually acquire the diagnosis of ADHD approximately 70 to 80% of the time. There is also an increased incidence of other diagnoses found to co-occur with ADHD, including antisocial personality disorder, oppositional defiant disorder, conduct disorder and drug abuse. Symptoms of ADHD are now said to persist into adulthood in 30 to 70% of cases, although this “discovery” was also in large part spearheaded by the pharmaceutical industry and had little to do with academic research. As a result of the foregoing, current “knowledge” of ADHD has been so corrupted by pharmaceutical industry influence that it is difficult to know where scientific fact begins and the business interests of industry end.

As first described in the DSM, ADHD was originally applied to a triad of symptoms (attention deficit, hyperactivity, and problems with impulse control), to which distractibility was later added. The diagnosis was thereafter somewhat superficially subdivided into those individuals thought to be more affected by the attention deficit aspects, versus those primarily affected by the hyperactivity and impulsivity, versus those felt to have the combination of all these symptom classes. In addition, it was noted that symptoms should appear before age 7 and cause significant difficulty in social or school or family endeavors.

There appears to be increasing evidence that the brain deficit in young children is centered on the basal ganglia, whereas over the course of a lifetime, the deficit seems to gradually involve more brain areas in and around the prefrontal cortex. However, the neurobiology of ADHD is complex, and remains a subject of speculation.

Evidence for the inheritability of ADHD has emerged in recent years, and this has been seen across family, adoption and twin studies. In monozygotic twins, the heritability appears to have been estimated at between 60 and 95%. In addition, polymorphisms have been discovered involving multiple dopamine receptors, further shoring up the genetic basis of the disorder. Indeed, ADHD now appears to be a number of related disorders of these same dopamine pathways, helping to explain to a degree the heterogeneity of the condition's presentation. Neuroimaging studies have also been able to gradually construct a description of an illness which involves abnormalities in total brain volume, as well as various changes found in the frontal lobes, basal ganglia, corpus callosum and cerebellum. These neurobiological abnormalities help to establish the limited understanding the academic world has been able to establish of an illness with core symptoms of inattention, distractibility, impulsivity, and motoric hyperactivity which are associated with abnormalities in these very neurological pathways and neurotransmitter systems.

There also is great confusion as to whether ADHD can be acquired (or at least aggravated by) developmental factors. First to be considered is the likelihood that such an inherited illness is likely to be found in at least one of the parents of an affected child, raising the question whether parenting could also be negatively affected with obvious deleterious results in the child's course. This could easily be imagined, since the parent's own attention and executive defects could well result in a chaotic home environment which could strongly affect the child's development.

To add to the complexity of how development can worsen a child's genetically inherited propensity for ADHD, the American Academy of pediatrics published a study indicating that the incidence of ADHD appears to be quite influenced by exposure to screen media and, most significantly, television. The chief results of the study were that the earlier the child started watching TV and the more hours watched especially (at early ages), the more likely a diagnosis of ADHD was to be reached when the child reached the age of 7. This was probably one of the first attempts to quantify the effects of the increasing abnormality of the modern day “typical childhood.” The fact is that evolution has maintained many constants in the development of children over hundreds of millennia, and these environmental constants are now being sacrificed (increasingly quickly, carelessly and even violently) in modern western culture. This study was rather revolutionary in that it demonstrated that something thought to be benign could quite possibly prove etiological in the causation or aggravation of a psychiatric syndrome such as ADHD. It has since come to be rather well known that television induces a trance-like state, especially in younger children, and that this state seems to correlate with a number of subsequent long term deleterious results. In this way, the television watching of children (which had for decades, since the arrival of TV in the 1950s, been pretty much taken for granted by the American populace to be a relatively harmless aspect of childhood), was finally quantitatively examined as having a possible deleterious influence on childhood development. What has also been learned since this study is that this trance-like state is notable for its increase in the theta wave content of brain waves, the very waves that make a child sleepy, groggy or suggestible.

The American Academy of Pediatrics (AAP) quickly released their warnings to the public in regards to television watching by the very young. In particular, the AAP warned the public to keep children entirely off of television before the age of four, and to thereafter strictly limit it in older children (e.g., to an hour a day for five-year-olds, and slightly more for six-year-olds).

This remarkable research by the AAP influenced subsequent efforts by the neurofeedback community following up on this important information. It also influenced other contemporary discoveries in the neurofeedback field, including the use by Dr. Joel Lubar of quantitative EEG (QEEG) to accurately calculate that relative fractions of the brain's EEG, or brainwave output, composed of theta waves and beta waves. It was found that the theta wave output divided by the beta wave output (the so-called “theta to beta ratio”, or θ/β) was an accurate predictor of ADHD. The ratio θ/β was found to be significantly elevated in ADHD students as compared to normal students of the same age. This implies that the brain of so-called ADHD children actually have a propensity to fall into a sort of semi-sleep state, and that a significant part of the so-called “hyperactivity” these students which showed in class (but did not show during play outdoors, for example) could be understood as simply the efforts on the part of the child to remain awake in front of the child's peers when constrained to sit still for hours in a chair. This understanding lead to the development of significant neurofeedback treatment modalities which were found to quickly decrease theta waves while also increasing beta, quite effectively ending the child's so called “ADHD” entirely without the need for medication.

Interesting research was also done by Teicher et al. in 1996 using infrared motion analysis, which found that so-called ADHD children also spent 66% less time immobile then normal. This was most notable when the children were required to sit still, although these same children were no more active than normal children if allowed to play. However, the concept that children have always played, and that play is actually an intrinsic and utterly necessary part of their nervous system's ability to achieve and maintain homeostasis, appears to have been overlooked.

Of course, it is important to remember that one of the main people involved in the re-creation of the American public education system in the 1920s and 30s was Henry Ford, who designed the new “improved” public school in exactly the same manner that he designed his Ford model T plants. In particular, in his design, each child moved along an invisible assembly line, surrounded by other children of identical age, learning identical courses at identical times, in a manner wholly analogous to the way automobile assembly workers were putting bolts and screws on automobiles moving through Detroit's assembly lines.

What needs to be considered is that, whereas it was originally claimed that inattention is usually due to a failure to sustain interest in tasks the child found boring or not challenging, that view completely failed to take into consideration the possibility that the child might quite simply be trying to remain awake while being forced into what is evolutionarily a very unnatural activity (i.e., being required to remain for hours at a time sitting still in a chair, in a long row of chairs, surrounded by other children the child would rather be playing with).

Another very significant problem discovered in the 1980s was that abused children who have developed symptoms consistent with PTSD were also frequently found to subsequently meet criteria for ADHD, and that this development appeared to occur in at least approximately ¼ of cases of PTSD in children. Here, it is exceedingly important to note just how inadequate the diagnosis of PTSD in DSM-V or DSM-IV has been in its failure to diagnose a very significant number of abused children as victims of abuse (a form of trauma). Actually, the DSM's PTSD diagnosis appears very likely to have left out the majority of nonlethally abused children and therefore, if the findings necessary to diagnose ADHD were present in 25% of PTSD diagnosed children, then the findings necessary to diagnose ADHD were also very possibly also present in 25% of the much larger group of undiagnosed developmental trauma disorder children. Furthermore, it was discovered in 1996 by Teicher et al. that 38% of hospitalized abused children also met criteria for ADHD.

It is also notable that significant co-morbidities are frequently observed whenever ADHD is diagnosed, such as Conduct Disorder (which can occur in 40 to 70% of ADHD cases). Learning Disorders are also very common, as is so called Oppositional Defiant Disorder. Again, it is notable that such “comorbidities” may actually be very poor diagnoses for trauma. As a result, these cases would likewise also possibly meet criteria for developmental trauma disorder, while at the same time failing to meet the criteria necessary for classic PTSD.

It is also notable that there are equally significant numbers of missed TBI cases, since head trauma often goes originally undiagnosed and is then forgotten about for all intents and purposes. One of the most frequent long term consequences of TBI is the frontal lobe damage resulting in various forms of attention deficit. If the head injury is not searched for during original diagnosis, these children and adolescents are often simply later identified as “ADHD” and are prescribed stimulants, even though other modalities (in particular, QEEG guided neurofeedback and comprehensive TBI care) would often give a far better result.

It is also notable that the chronic sleep deprivation of adolescents is epidemic, and may result in symptoms quite similar to the inattention and distractibility associated with ADHD. This is no doubt influenced, in part, by the social world of today's adolescents and their propensity to socialize at all hours. However, it also needs to be understood that sleep disorders are the rule in the ADHD population, and the sleep difficulties of ADHD children and adolescents have been found to increase their main ADHD symptoms synergistically. This often results in careless increases in dosages of stimulants, which then increase the sleep inadequacy in an iterative fashion. Furthermore, latent bipolar disorder has all too often also been discovered in “ADHD” children and adolescents, frequently due to these very stimulants triggering manic episodes that often result in hospitalization.

It is also notable that the amphetamines used to “control” ADHD symptoms are just as addictive as amphetamines abused on the street. Even when the child or adolescent is lucky enough to not get addicted, the side effects of stimulants are very frequently quite significant. These side effects, which may include insomnia, irritability, anger, rage and marked changes in personality, may severely affect the parent-child relationship, and may adversely affect the child's entire family. Furthermore, the use of amphetamines may result in a significant loss of appetite and subsequent weight loss, which may lead to or worsening eating disorders in children and adolescents. Tragically, these eating disorders and their intertwined stimulant addictions, which are more common in young females, may also become lifelong and life threatening. Growth may also be affected, and the resulting stunted growth may result in further issues to the child facing it.

There is also very significant and recent data showing that mindfulness, yoga, exercise and meditation have all been found to improve ADHD without drugs. Neurofeedback seems quite capable, in the great majority of ADHD cases, of simply ending the problem, at least insofar as academic performance is concerned, and of doing so at acceptable total cost in time and money. The danger of stimulant use is potentially catastrophic in the not inconsiderable number of cases of ADHD or faked “ADHD”, and is quite significant in all remaining indeterminate cases.

ADHD appears to be a neurobiological disorder affecting children, adolescents and adults with a prevalence somewhere in the 3-5% range. There is now convincing heritability and genetic data, neuroimaging data and quantitative EEG data which all point to a heritable condition affecting attentional, motoric and impulse control aspects of the brain. This disorder then appears, in roughly half of correctly diagnosed child and adolescent cases, to go on to significantly affect the patient's adult life. These bona fide ADHD patients need and deserve help, especially in building the skills and mental strengths which result in adequate or at least significantly improved attention, and this should be accomplished to whatever extent possible without the use of stimulants and amphetamines. This approach should be promoted on a national scale, and the building of attention should begin in early childhood (as, for example, by preventing screen abuse by those responsible for guiding small children, and by directing such children towards more traditional forms of behavior and interaction with others, especially play with other children).

Unfortunately, this disorder has also been massively promoted and oversold by the pharmaceutical industry to an alarming extent, although there is blame to be shared by government, school administrations and physicians. It should also be noted that the great majority of these prescriptions are not even written by psychiatrists or child psychiatrists, although these two groups also share some of the blame in this epidemic of overdiagnosis and resulting stimulant overuse. However, ADHD should be entirely reexamined as a clinical entity to be treated by first doing no harm to the patient wherever possible. This will likely require a significant national effort by such organizations as the NIMH and academic medical research centers, and should include strict rules for the prevention of such “researchers” from ever profiting from the sale of this diagnosis and its attendant stimulant prescriptions.

Reasonable efforts should be made from this point forward to find all possible nonmedication modalities for helping legitimate attention deficit cases, employing both the most ancient and the most up to date modalities available. Light therapy also would appear promising in this regard, at least insofar as it can likely improve sleep and mood problems. It needs to be kept in mind, however, that a significant fraction of true ADHD cases have thus far unexpressed or unsuspected bipolar disorder in them, which bipolarity can also be brought on by stimulants or insomnia. However, it is currently not known what other effects on early bipolar disorder might result from light therapy. Therefore, any attempts to treat ADHD with light therapy should proceed with all due caution in this regard.

### D. Possible Modifications or Permutations

Various aspects of the systems and methodologies described herein have been described above with respect to the particular, non-limiting embodiments disclosed herein. It will be appreciated that these various aspects may be employed in various combinations (including various sub-combinations) or permutations in accordance with the teachings herein.

For example, while the use of light sources which emit at wavelengths in the red, infra-red and blue-turquoise regions of the spectrum are preferred, and the use of light sources which emit at about 470 nm, 670 nm and 870 nm are especially preferred, it will be appreciated that the devices and methodologies disclosed herein may utilize various other frequencies or wavelengths of electromagnetic radiation to achieve desired physiological or psychological effects. These wavelengths or frequencies may be selected, for example, from the visible, infrared or ultraviolet regions of the electromagnetic spectrum.

Similarly, in a preferred mode of operation, the intensities of one or more of these light sources are made to oscillate or flicker in the theta or gamma frequency band during at least a portion of a therapy session. However, embodiments are possible in which the light sources are made to oscillate or flicker at other frequencies, or in which the light sources (or elements thereof) operate in a manner which is not time varying. Embodiments are also possible in which the light sources are made to oscillate or flicker at harmonics of the foregoing frequencies.

While the embodiment of FIGS. 1-8 is a preferred embodiment of the light therapy unit described herein, it will be appreciated that light therapy units of various shapes, configurations, layouts and functionalities may be utilized in the practice of the methodologies disclosed herein, and these light therapy units may be provided with various accessories.

For example, in some embodiments, light therapy units may be utilized that are adapted to illuminate one or more inner surfaces of a subject's oral cavity. In such embodiments, a light therapy unit utilized for this purpose may be fashioned as a standalone device, while in other embodiments, such a light therapy unit may be fashioned as an accessory to a main light therapy unit which is utilized to illuminate the outer surfaces of a subject's head. In embodiments of the latter type, the accessory may be adapted to communicate with the main light therapy unit such that the accessory is controlled by, or acts in concert with, the main light therapy unit.

In some instances of embodiments of a light therapy unit adapted to illuminate one or more inner surfaces of a subject's oral cavity, the light therapy unit may be equipped with a mouth guard which is in optical communication with a light source by way of a suitable light guide, and which distributes light received from the light source in a suitable manner. In some cases, the mouthguard may be customized to the user. By way of example but not limitation, such a mouth guard may be adapted to direct suitable wavelengths of light to various surfaces of the oral cavity of a subject, including the teeth, gums, upper or lower mouth, and throat. The mouth guard, light guide or portions thereof may be equipped with suitable materials that specularly or diffusely transmit or reflect incident radiation in one or more directions. In addition to their possible use in treating physiological or psychological conditions, these embodiments may offer additional benefits such as, for example, the treatment or prevention of gingivitis and other bacterial infections.

In some embodiments of the devices disclosed herein, measures may be taken to ensure that the light therapy is applied to only specific parts of the user's body. For example, in some embodiments, the aforementioned light therapy unit which is adapted to illuminate one or more inner surfaces of a subject's oral cavity may be used by itself such that only these surfaces are exposed to the light therapy. Similarly, in some embodiments, the user may be equipped with glasses or goggles such that the user's eyes or optical nerves are not exposed to the light therapy, or such that the light therapy is concentrated on the user's eyes or optical nerves. In still other embodiments, an optical pad or other suitable means may be utilized to apply light therapy only to the back of a user's neck.

Preferred embodiments of the devices disclosed herein are adapted to allow the user to lie down or otherwise assume a state of repose during a light therapy session. Such embodiments may include, for example, a pillow or one or more deformable pads which support the user's head during light therapy. Here, it is notable that many other devices in the art which are designed for light therapy require the user to remain in a sitting or standing position for the duration of the therapy.

In some embodiments of the devices disclosed herein, the device may be equipped with a suitable controller, which may be wireless or wired. The controller may be programmable or pre-programmed, and may be equipped with suitable programming instructions (which may include an operating system) recorded in a tangible, non-transient medium that cause the light therapy device to operate in various modes or to perform various functions. These modes or functions may be selected or optimized for the treatment of various portions of a subject's body, or for the treatment of particular physiological or psychological conditions.

Various parameters (and ranges of these parameters) may be utilized in the light therapy devices and methodologies disclosed herein. These include, without limitation, wavelength, frequency, energy, fluence, power, irradiance, intensity, pulse mode, treatment duration, and repetition. These parameters and their values may be selected to treat a subject for certain psychological or physiological conditions, to lessening the severity or effects of these conditions, and/or to preventing the occurrence of these conditions. These conditions include, but are not limited to, traumatic brain injury, opioid addiction, depression (and more specifically, clinical depression or major depression), and developmental trauma.

It will be appreciated that the light therapy units disclosed herein, and the components thereof, may be equipped with suitable optical elements to achieve various purposes. Such optical elements (or portions thereof) may be diffusely or specularly reflective or transmissive. Suitable optical elements may include, but are not limited to, reflective elements, polarizers, color shifting elements, filters, light guides (including, without limitation, optical fibers, light pipes and waveguides), prismatic elements, lenses (including Fresnel lenses), and lens arrays.

In preferred embodiments of the systems and methodologies disclosed herein, one or more audio tracks or audio files may be provided that may be modulated, coordinated and/or synchronized with the plurality of LEDs or the light emitted therefrom. Preferably, the audio tracks or audio files include sound that is modulated, coordinated and/or synchronized with the LEDs or the light emitted therefrom at a frequency within the range of about 20 Hz to about 60 Hz, and more preferably within the range of about 35 Hz to about 45 Hz. The audio tracks or files may be selected to achieve a desired physiological or psychological effect in the user, either alone or in combination with the light therapy.

One skilled in the art will further appreciate that the systems and methodologies disclosed herein may be used not only to treat various physiological or psychological conditions, but to prevent them from occurring in the first place. For example, these systems and methodologies may be adapted to prevent the onset of depression, PTSD, ADHD, opioid addiction, or conditions resulting from traumatic brain injury, or of conditions which might otherwise result from the foregoing.

One skilled in the art will further appreciate that the optimal parameters for a light therapy session may depend on a variety of factors including, but not limited to, the condition being treated (or prevented), the physiological or psychological state of the user, the user's biometrics, and other such factors. In some use cases, selection of these parameters may be made by, or in coordination with, a physician, a psychiatrist, or other healthcare provider. These parameters may include, but are not limited to, the wavelengths of light to be utilized, the audio tracks or files to accompany the light therapy, the frequencies of oscillation utilized for the intensity in any of the wavelengths or light or sound, the portions of the user's head or body to be exposed to the light therapy, and the duration of the treatment.

The above description of the present invention is illustrative, and is not intended to be limiting. It will thus be appreciated that various additions, substitutions and modifications may be made to the above described embodiments without departing from the scope of the present invention. Accordingly, the scope of the present invention should be construed in reference to the appended claims. It will also be appreciated that the various features set forth in the claims may be presented in various combinations and sub-combinations in future claims without departing from the scope of the invention. In particular, the present disclosure expressly contemplates any such combination or sub-combination that is not known to the prior art, as if such combinations or sub-combinations were expressly written out.

What is claimed is:

1. A method for treating a subject for depression, comprising:
diagnosing the subject as suffering from clinical depression or major depression;
providing a light therapy unit comprising (a) a chassis, (b) a plurality of LEDs disposed on said chassis, and (c) a controller which controls the operation of said LEDs; and
operating the plurality of LEDs at a plurality of distinct wavelengths of light such that the light emitted by the LEDs impinges on the subject, and such that the intensity of at least one of the plurality of distinct wavelengths of light is modulated at a frequency within the range of about 20 Hz to about 60 Hz.

2. The method of claim 1, wherein the intensity of at least one of the plurality of distinct wavelengths of light is modulated at a frequency within the range of about 35 Hz to about 45 Hz.

3. The method of claim 1, wherein the intensity of at least one of the plurality of distinct wavelengths of light is modulated at a frequency of about 40 Hz.

4. The method of claim 1, wherein said plurality of distinct wavelengths of light include first, second and third distinct wavelengths of light.

5. The method of claim 1, wherein said plurality of LEDs includes a first, second and third plurality of LED lights disposed on said chassis, and wherein said first, second and third plurality of LED lights operate, respectively, at first, second and third distinct wavelengths.

6. The method of claim 1, wherein said chassis has an opening therein which accommodates the head of the subject, and further comprising:
placing the head of the subject in the opening.

7. The method of claim 1, wherein the plurality of LEDs are operated such that the light emitted by the LEDs impinges on the head of the user.

8. The method of claim 1, wherein one of said plurality of distinct wavelengths of light is in the red region of the spectrum.

9. The method of claim 1, wherein one of said plurality of distinct wavelengths of light is in the near-infrared region of the spectrum.

10. The method of claim 1, wherein one of said plurality of distinct wavelengths of light is in the blue/turquoise region of the spectrum.

11. A method for treating a subject suffering from a traumatic brain injury, comprising:
diagnosing the subject as suffering from a traumatic brain injury;
providing a light therapy unit comprising (a) a chassis, (b) a plurality of LEDs disposed on said chassis, and (c) a controller which controls the operation of said LEDs; and
operating the plurality of LEDs at a plurality of distinct wavelengths of light such that the light emitted by the LEDs impinges on the subject, and such that at least one of the plurality of distinct wavelengths of light is modulated at a frequency within the range of about 20 Hz to about 60 Hz.

12. The method of claim 11, wherein the intensity of at least one of the plurality of distinct wavelengths of light is modulated at a frequency within the range of about 35 Hz to about 45 Hz.

13. The method of claim 11, wherein the intensity of at least one of the plurality of distinct wavelengths of light is modulated at a frequency of about 40 Hz.

14. The method of claim 11, wherein said plurality of distinct wavelengths of light include first, second and third distinct wavelengths of light.

15. The method of claim 11, wherein said plurality of LEDs includes a first, second and third plurality of LED lights disposed on said chassis, and wherein said first, second and third plurality of LED lights operate, respectively, at first, second and third distinct wavelengths.

16. The method of claim 11, wherein said chassis has an opening therein which accommodates the head of the subject, and further comprising:
placing the head of the subject in the opening.

17. The method of claim 11, wherein the plurality of LEDs are operated such that the light emitted by the LEDs impinges on the head of the user.

18. The method of claim 11, wherein one of said plurality of distinct wavelengths of light is in the red region of the spectrum.

19. The method of claim 11, wherein one of said plurality of distinct wavelengths of light is in the near-infrared region of the spectrum.

20. The method of claim 11, wherein one of said plurality of distinct wavelengths of light is in the blue/turquoise region of the spectrum.

21. A method for treating a subject suffering from an opioid addiction, comprising:
diagnosing the subject as suffering from an opioid addiction;
providing a light therapy unit comprising (a) a chassis, (b) a plurality of LEDs disposed on said chassis, and (c) a controller which controls the operation of said LEDs; and
operating the plurality of LEDs at a plurality of distinct wavelengths of light such that the light emitted by the LEDs impinges on the subject, and such that at least one of the plurality of distinct wavelengths of light is modulated at a frequency within the range of about 20 Hz to about 60 Hz.

22. The method of claim 21, wherein the intensity of at least one of the plurality of distinct wavelengths of light is modulated at a frequency within the range of about 35 Hz to about 45 Hz.

23. The method of claim 21, wherein the intensity of at least one of the plurality of distinct wavelengths of light is modulated at a frequency of about 40 Hz.

24. The method of claim 21, wherein said plurality of distinct wavelengths of light include first, second and third distinct wavelengths of light.

25. The method of claim 21, wherein said plurality of LEDs includes a first, second and third plurality of LED lights disposed on said chassis, and wherein said first, second and third plurality of LED lights operate, respectively, at first, second and third distinct wavelengths.

26. The method of claim 21, wherein said chassis has an opening therein which accommodates the head of the subject, and further comprising:
placing the head of the subject in the opening.

27. The method of claim 21, wherein the plurality of LEDs are operated such that the light emitted by the LEDs impinges on the head of the user.

28. The method of claim 21, wherein one of said plurality of distinct wavelengths of light is in the red region of the spectrum.

29. The method of claim 21, wherein one of said plurality of distinct wavelengths of light is in the near-infrared region of the spectrum.

30. The method of claim 21, wherein one of said plurality of distinct wavelengths of light is in the blue/turquoise region of the spectrum.

31. A method for treating a subject suffering from ADHD, comprising:
diagnosing the subject as suffering from ADHD;
providing a light therapy unit comprising (a) a chassis, (b) a plurality of LEDs disposed on said chassis, and (c) a controller which controls the operation of said LEDs; and
operating the plurality of LEDs at a plurality of distinct wavelengths of light such that the light emitted by the LEDs impinges on the subject, and such that at least one of the plurality of distinct wavelengths of light is modulated at a frequency within the range of about 20 Hz to about 60 Hz.

32. The method of claim 31, wherein the intensity of at least one of the plurality of distinct wavelengths of light is modulated at a frequency within the range of about 35 Hz to about 45 Hz.

33. The method of claim 31, wherein the intensity of at least one of the plurality of distinct wavelengths of light is modulated at a frequency of about 40 Hz.

34. The method of claim 31, wherein said plurality of distinct wavelengths of light include first, second and third distinct wavelengths of light.

35. The method of claim 31, wherein said plurality of LEDs includes a first, second and third plurality of LED lights disposed on said chassis, and wherein said first, second and third plurality of LED lights operate, respectively, at first, second and third distinct wavelengths.

36. The method of claim 31, wherein said chassis has an opening therein which accommodates the head of the subject, and further comprising:
placing the head of the subject in the opening.

37. The method of claim 31, wherein the plurality of LEDs are operated such that the light emitted by the LEDs impinges on the head of the user.

38. The method of claim 31, wherein one of said plurality of distinct wavelengths of light is in the red region of the spectrum.

39. The method of claim 31, wherein one of said plurality of distinct wavelengths of light is in the near-infrared region of the spectrum.

40. The method of claim 31, wherein one of said plurality of distinct wavelengths of light is in the blue/turquoise region of the spectrum.

41. A method for treating a subject suffering from PTSD, comprising:
diagnosing the subject as suffering from PTSD;
providing a light therapy unit comprising (a) a chassis, (b) a plurality of LEDs disposed on said chassis, and (c) a controller which controls the operation of said LEDs; and
operating the plurality of LEDs at a plurality of distinct wavelengths of light such that the light emitted by the LEDs impinges on the subject, and such that at least one of the plurality of distinct wavelengths of light is modulated at a frequency within the range of about 20 Hz to about 60 Hz.

42. The method of claim 41, wherein the intensity of at least one of the plurality of distinct wavelengths of light is modulated at a frequency within the range of about 35 Hz to about 45 Hz.

43. The method of claim 41, wherein the intensity of at least one of the plurality of distinct wavelengths of light is modulated at a frequency of about 40 Hz.

44. The method of claim 41, wherein said plurality of distinct wavelengths of light include first, second and third distinct wavelengths of light.

45. The method of claim 41, wherein said plurality of LEDs includes a first, second and third plurality of LED lights disposed on said chassis, and wherein said first, second and third plurality of LED lights operate, respectively, at first, second and third distinct wavelengths.

46. The method of claim 41, wherein said chassis has an opening therein which accommodates the head of the subject, and further comprising:
placing the head of the subject in the opening.

47. The method of claim 41, wherein the plurality of LEDs are operated such that the light emitted by the LEDs impinges on the head of the user.

48. The method of claim 41, wherein one of said plurality of distinct wavelengths of light is in the red region of the spectrum.

49. The method of claim 41, wherein one of said plurality of distinct wavelengths of light is in the near-infrared region of the spectrum.

50. The method of claim 41, wherein one of said plurality of distinct wavelengths of light is in the blue/turquoise region of the spectrum.
